(19) United States
(12) Patent Application Publication
(10) Pub. No.: US 2009/0274576 A1
(43) Pub. Date: Nov. 5, 2009

(54) System and method for container sterilization using UV light source

(76) Inventor: Barry Ressler, Danbury, CT (US)

Correspondence Address: MCCARTER & ENGLISH, LLP STAMFORD, FINANCIAL CENTRE , SUITE 304A, 695 EAST MAIN STREET, STAMFORD, CT, 06901-2138, US

(21) Appl. No.: 11/654,475

(22) Filed: Jan. 17, 2007

Related U.S. Application Data
(60) Provisional application No. 60/759,946, filed on Jan. 18, 2006.

(51) Int. Cl. A61L 2/10; A61L 2/08

(52) U.S. Cl. 422/24; 422/22; 422/186; 422/186.3

(57) ABSTRACT

Packaging materials and containers may be treated with sterilization dosages internally and externally using monochromatic, continuous wave, high-intensity, incoherent light in single and/or multiple light source configurations. The treatment systems and methods preserve physical and performance properties of the packaging/container while achieving a desired level of sterilization. The sterilized materials may be adapted for extended shelf life (ESL) products. The disclosed treatment systems and methods may also be used for sterilization of food and beverage products (either prepackaged or post packaged), medicines, pharmaceuticals, vitamins, infusion products, clinical and/or non-clinical solutions and systems, enteral and/or parenteral solutions and systems, and the like.

### CROSS-REFERENCE TO RELATED APPLICATION(S)

The present application claims the benefit of a co-pending provisional patent application entitled “System and Method for Product Container Sterilization Using UV Light Source,” which was filed on Jan. 18, 2006 and assigned Ser. No. 60/759,946. The entire contents of the foregoing provisional patent application are incorporated herein by reference.

### BACKGROUND

1. Technical Field

The present disclosure is directed to system(s) and method(s) for sterilization of packaging and/or containers for liquid and/or solid products, e.g., food and beverage products, using UV light source(s). More particularly, the present disclosure is directed to system(s) and method(s) for the non-thermal and non-chemical sterilization of packaging and/or containers that involves introduction of a sterilizing energy source through a lid opening and/or external delivery of such sterilizing energy. Exemplary packaging materials that may be subjected to the sterilizing energy disclosed herein include, inter alia, polyethyleneterephthalate (PET), polyethylenenapthalene (PEN), polyethylene (PE), polypropylene (PP), paperboard (e.g., cartons adapted for aseptic packaging applications, gable top cartons and the like), aluminum foil laminated bags/packages adapted for aseptic packaging applications and glass packaging materials.

2. Background Art

Sterilization is generally defined as the complete destruction of all organisms, including a large number of highly resistant bacterial endospores. A host of sterilization techniques have been developed to address specific sterilization needs. Typical sterilization techniques include the use of moist heat from a steam autoclave, ethylene oxide gas sterilizing techniques, dry heat techniques, and newer chemical sterilizers.

Steam sterilization is widely used and is generally viewed as a relatively cost-effective sterilization technique. Steam sterilization techniques employing an autoclave are recognized as efficient, simple, and relatively cost-effective approaches for destroying relevant organisms. However, certain components (e.g., medical device/instrumentation components and accessories) cannot endure the extremes of heat and pressure. For example, steam and pressure are known to risk damage to rubber, Lexan® polycarbonate components, and other synthetic materials, and the use of steam autoclave techniques for anesthesia equipment is generally not recommended, unless the treatment method is specifically recommended by the manufacturer.

Ethylene oxide is acceptable for many materials used in manufacturing medical devices and the like, including the reusable components of anesthesia machines, ventilators, and monitors. However, it is generally inappropriate to place these entire systems in an ethylene oxide chamber. In addition, polystyrene component parts generally should not be exposed to ethylene oxide gas. Ethylene oxide sterilization employs a powerful poisonous fumigant gas, and therefore mandates an appropriate means of aeration to remove residual gas. Workers exposed to ethylene oxide are required to comply with all procedures specified by OSHA and the EPA. Alternative chemical treatment techniques include the use of hydrogen peroxide and peroxyacetic acid with buffers and low heat.

With reference to the patent literature, a sterilization technique was disclosed in U.S. Pat. No. 5,786,598 to Clark et al., entitled “Sterilization of Packages and Their Contents Using High-Intensity, Short-Duration Pulses of Incoherent, Polychromatic Light in a Broad Spectrum.” As noted in the title, the Clark '598 patent involves the use of high-intensity, short-duration pulses of incoherent, polychromatic light in a broad spectrum to sterilize product containers and deactivate microorganisms therein. The Clark '598 patent proposes “the deactivation of microorganisms within parenteral and/or enteral solutions and packages or within contact lens solutions and packages and/or ophthalmic solutions and packages.” [See col. 1, lines 11-20.] The use of short-duration pulses of incoherent, polychromatic light in a broad spectrum, as disclosed in the Clark '598 patent, is believed to be ineffective and/or unacceptable for at least some aspects of the proposed applications.

Despite efforts to date, a need remains for system(s) and/or method(s) for use in sterilizing a broad range of packaging/container systems, e.g., polymer-based packages/containers, aluminum-based packages/containers and glass-based packages/containers, wherein such sterilization regimen achieves a desired sterilization level without negatively affecting the physical properties of the package/container and/or the underlying material structure and packaging utility thereof. A need also exists for systems and methods for sterilizing products (e.g., food products such as meat and poultry, enteral and/or parenteral solutions and systems, and the like) that fill the containers, whether positioned within or external to the packaging container, wherein such treatment regimen achieves a desired sterilization level without negatively affecting the physical properties and/or the efficacy of the underlying product(s)/system(s).

### SUMMARY OF THE DISCLOSURE

According to exemplary embodiments of the present disclosure, packaging materials and/or containers may be treated internally and/or externally using monochromatic, continuous wave, high-intensity, incoherent light in single and/or multiple light source configurations. The disclosed treatment system(s) and method(s) advantageously preserve physical and performance properties of the product/system while achieving a desired level of sterilization. In advantageous applications of the disclosed system(s) and method(s), the sterilized materials (e.g., packaging materials and/or containers) are adapted for extended shelf life (ESL) products. The disclosed treatment system(s) and method(s) may also be used for sterilization of food and beverage products (either prepackaged or post packaged), medicines, pharmaceuticals, vitamins, infusion products, clinical and/or non-clinical solutions and systems, enteral and/or parenteral solutions and systems, and the like.

More particularly, according to exemplary embodiments of the present disclosure, sterilization of packaging/container products and systems is advantageously achieved using monochromatic, continuous wave, high-intensity, incoherent light in single and/or multiple light source configurations. The disclosed treatment system(s) and method(s) advantageously achieve a desired sterilization level without negatively affecting the physical properties and/or the efficacy of the underlying product(s)/system(s). An advantageous approach to the sterilization of product packaging and/or containers, including packaging/container systems that include heat sensitive materials is disclosed herein. The disclosed sterilization systems and methods have wide ranging applicability, and may employed to sterilize packaging/container systems through the delivery of sterilizing energy, whether within or external to the packaging and/or container system. According to exemplary embodiments of the present disclosure, the disclosed sterilization systems and methods are effective in inactivating viral and bacterial microorganisms without physical or performance-related damage to the treated product packaging/container.

More specifically, a single or multiple array of light sources may be employed according to the present disclosure to deliver monochromatic germicidal light at radiance levels of about 200 mW/cm² to 600 mW/cm² to deactivate multiple organisms. The germicidal light is advantageously delivered at a substantially ambient temperature so as to avoid potential temperature-related damage to the packaging/container system. According to exemplary embodiments of the present disclosure, the germicidal light may be generated and delivered at substantially discrete wavelengths, e.g., wavelengths of 193 nm; 222 nm; 248 nm; 282 nm; 308 nm and 354 nm. The light wavelength may be advantageously controlled to +/−5 nm.

The disclosed sterilization treatment regimen may be undertaken in a batch, semi-batch or continuous mode. In an exemplary embodiment of the present disclosure, target packaging/container product(s) are treated continuously or semi-continuously by positioning the packaging/container units on a moving belt or other indexing mechanism, such that the packaging/container units are moved in an indexed fashion to a treatment zone. For example, the packaging/container units may be indexed in preset numbers, e.g., sets of six, ten, twelve or the like, and thereby positioned in substantial alignment with a sterilizing light source. When positioned in a predetermined position, a germicidal element (e.g., a quartz light pipe) according to the present disclosure interacts with the internal geometry of the container/packaging to achieve a sterilization effect.

Thus, in an exemplary embodiment of the present disclosure, a germicidal element (e.g., a quartz light pipe) is adapted to be introduced through the neck of a package/container so as to introduce germicidal light energy to the interior of such package/container. Of note, the germicidal element is advantageously adapted to deliver germicidal light energy over a longitudinal zone of treatment, based on the vertical/longitudinal movement of the germicidal element downward into the package/container and then upward out of the package/container. Thus, the germicidal element achieves essentially two germicidal passes with respect to the side wall(s) of the package/container. Moreover, the disclosed germicidal element achieves a horizontally-dispersed treatment effect based, at least in part, on the geometry of the germicidal element, e.g., the distal tip geometry of a disclosed quartz light pipe.

According to exemplary embodiments of the present disclosure, a substantially uniform disc (circumference) of germicidal dosage is twice delivered to all internal surfaces of the package/container, prior to the package/container entering the fill and cap stage. The rate at which the package(s)/container(s) are moved past the light source(s) may be adjusted so as to achieve the desired energy treatment level, e.g., based on a desired residence time of the germicidal element within each package/container. In batch/semi-batch implementations, the treatment time may be varied to achieve the desired energy treatment level. As noted below, additional processing parameters may be controlled/modified so as to affect a desired sterilization result according to the present disclosure and such processing parameters may be adjusted/selected (either alone or in combination with the rate/residence time of the germicidal element within the package/container) to achieve desired sterilization result(s).

Thus, the intensity of the monochromatic light source(s) that are employed according to the sterilization system(s) and/or method(s) of the present disclosure may be adjusted to achieve desired sterilization results. Each light source may include and/or interact with multiple germicidal elements. Individual germicidal elements may be advantageously spaced in accordance with various shapes and sizes of packages/containers, e.g., based on the indexing/spacing of the packages/containers in a predefined treatment zone. The individual germicidal elements may be operated at different energy intensities to achieve desired sterilization results, whether based upon or independent of container size, shape, color and/or material.

Light source intensity is generally selected according to the present disclosure based on treatment algorithm(s) for a single microorganism or suite (panel) of organisms/microorganisms. In typical treatment regimens, the panel of organisms includes, but is not limited to, Bacillus pumilus (spore former), Candida albican (yeast), lipid and non-lipid virus, Clostridium sporogenes (anaerobic spore former), Alicyclobacillus, Staphylococcus aureus (vegetative Gram positive), Pseudomonas aeruginosa (vegetative Gram negative), Aspergillus niger (filamentous fungi), Mycobacterium terrae, Porcine Parvo Virus (PPV and B19), Lysteria, and Salmonella. The sterilization treatment regimen and associated systems/apparatus disclosed herein are effective in treating packaging/containers of varying sizes, shapes and geometries. Thus, for example, the package and/or product container may be planar, convex, concave or an alternative geometry, e.g., a geometric combination of the foregoing geometries. The germicidal elements may be modified to achieve desired results. Thus, for example, partially coated optical surfaces may be employed, such coated surfaces being advantageously tuned to a desired monochromatic wavelength. The use of partially coated optical surfaces may be effective in generating light that satisfies spectral intensity requirements in excess of 500 mW/cm².

Additional features and functionalities associated with the disclosed sterilization system(s) and method(s) will be apparent from the detailed description which follows, particularly when viewed together with the figures appended hereto.

### BRIEF DESCRIPTION OF THE FIGURES

To assist those of ordinary skill in the art to which the present disclosure appertains in making and using the disclosed sterilization system(s) and method(s), reference is made to the appended figures, wherein:

FIG. 1 is a top view of an exemplary sterilization reactor with multiple output ports for the acceptance of/cooperation with light pipe delivery components for delivering monochromatic, continuous wave, high-intensity, incoherent light to packages/containers, e.g., polymer-based, cardboard and/or glass packages/containers, using a single light source, according to the present disclosure;

FIG. 2 is a schematic drawing of an exemplary quartz light pipe with a shaped end for the distribution of monochromatic, continuous wave, high-intensity, incoherent light and without a coating or reflective material, according to the present disclosure;

FIG. 3 is a schematic drawing of a plurality of light pipes interacting with packages/containers to deliver sterilizing energy thereto, according to an exemplary embodiment of the present disclosure; and

FIGS. 4a and 4b are views of the distal end of an exemplary light pipe according to the present disclosure.

### DETAILED DESCRIPTION OF EXEMPLARY EMBODIMENT(S)

According to the present disclosure, systems and methods for sterilization of packaging products/containers, including heat sensitive materials, whether internal or external to the packaging containers, are provided. These systems/methods are effective in inactivating viral and bacterial microorganisms without physical or performance-related damage to the treated packaging product/container. A single or multiple array of light sources delivers monochromatic germicidal light at irradiance levels of at least 200 mW/cm² to 600 mW/cm² to deactivate multiple organisms, preferably substantially at ambient temperature. According to exemplary embodiments of the present disclosure, discrete germicidal wavelengths are generated and delivered, e.g., wavelengths of 193 nm; 222 nm; 248 nm; 282 nm; 308 nm and 354 nm (+/−5 nm). The disclosed wavelengths are generally effective for use in sterilizing a range of materials, e.g., polyethyleneterephthalate (PET), polyethylenenapthalene (PEN), polyethylene (PE), polypropylene (PP), paperboard (e.g., aseptic cartons and gable top milk cartons), aluminum foil laminated aseptic bags, and glass packages/containers. In a further exemplary embodiment of the present disclosure, treatment of polymeric contact lens products (whether packaged or non-packaged) may be advantageously undertaken/achieved at wavelengths of 282 nm and 308 nm.

The disclosed sterilization treatment regimen may be undertaken in a batch, semi-batch or continuous mode. The application of monochromatic UV light using the disclosed light source(s) to inactivate viral and bacterial microorganisms in sterilizing contact lenses and/or packaging for contact lenses is a particularly attractive alternative to currently practiced sterilization methods, such as steam sterilization, because the disclosed UV radiation treatment is readily incorporated into an in-line (i.e., continuous or substantially continuous) process, in which the sterilization may be accomplished in a matter of seconds. In addition, the disclosed monochromatic UV light is effective for sterilization of heat sensitive materials without negatively affecting physical properties and/or performance attributes thereof. Additional performance features/functionalities associated with such polymer-based products (e.g., contact lenses) that were not feasible with conventional steam sterilization (e.g., because steam sterilization damaged or destroyed such features/functionalities) are potentially feasible using the disclosed monochromatic UV sterilization technique. A co-pending, commonly assigned patent application entitled “System and Method for Product Sterilization Using UV light Source” sets forth additional specifics as to contact lens-related sterilization techniques and results, which was filed on Feb. 11, 2005, and assigned Ser. No. 11/056,698. The foregoing application was published as US Patent Publication No. 2005/0173652 on Aug. 11, 2005, and the entire content of the foregoing application/publication is hereby incorporated by reference.

In an exemplary embodiment of the present disclosure, target product(s) and/or container-packaging product(s) are treated continuously (or semi-continuously) by positioning the product(s)/container(s) on a moving element that is indexed into and out of the internal structure of the container delivering uniform dosage to all internal surfaces for the duration of the indexed insertion and removal. A variety of structures and mechanisms may be used to transport products through the intermediate region while permitting UV radiation to reach the products for sterilization purposes, e.g., conveyor belts, rotating indexed machinery and/or tracks of various designs and constructions. The selection and implementation of appropriate conveyor/transport systems is well within the skill of persons skilled in the art. It is further expressly noted that transport systems may be incorporated in single light source implementations of the disclosed sterilization systems.

The rate at which the container(s) are moved in the germicidal light source(s) path in continuous or semi-continuous embodiments of the present disclosure may be adjusted so as to achieve the desired energy treatment level based, for example, on desired residence times in the treatment zone. Similarly, in batch/semi-batch embodiments, the treatment time may be varied to achieve the desired energy treatment level. Additional processing parameters may affect the sterilization procedure and may be adjusted/selected (either alone or in combination with the rate/residence time and/or other processing parameters) to achieve the desired energy delivery and resultant sterilization effect(s).

Thus, the intensity of the monochromatic light source(s) that are employed according to the sterilization system(s) and/or method(s) of the present disclosure and the design/operation of the light pipe transmission component(s) may be adjusted to achieve desired sterilization results. For example, in processing systems wherein alternative (hybrid construction components) of the package/container are presented, it may be necessary/desirable to incorporate reflective or coated elements onto or into the distal end (or distal region) of the disclosed light pipe. The incorporation of reflective or coated elements onto the distal end (or distal region) of the light pipe may be effective in dispersing, accentuating and/or filtering the light energy delivered by the light pipe, as will be apparent to persons skilled in the art. Similarly, geometric modifications to the distal end of the light pipe may be used to effect comparable modifications to light energy delivery parameters.

According to exemplary embodiments of the present disclosure, a control system may be advantageously associated with the light source(s) to control operating parameters thereof. A typical control system includes a processor that is programmed to operate the light sources at desired intensity levels and for desired period(s) of time. In the case of continuous treatment regimens, the control system may also advantageously be linked to the indexing system to control the rate at which products pass through the treatment region, e.g., based on the speed of the indexing/conveyor/transport system. A manual over-ride is typically provided, so as to permit an operator to adjust/modify treatment parameters on an as-needed basis.

Treatment parameters, e.g., light source intensity, are generally selected based on the treatment algorithm(s) for a single microorganism or suite (panel) of organisms/microorganisms. In typical treatment regimens, the panel of organisms includes, but is not limited to, Bacillus pumilus (spore former), Candida albican (yeast), lipid and non-lipid virus, Alicyclobacillus, Clostridium sporogenes (anaerobic spore former), Staphylococcus aureus (vegetative Gram positive), Pseudomonas aeruginosa (vegetative Gram negative), Aspergillus niger (filamentous fungi), Mycobacterium terrae, Porcine Parvo Virus (PPV and B19), Lysteria, and Salmonella. Additional and/or alternative organisms may be taken into consideration, in whole or in part, in developing and implementing an appropriate treatment regimen, as will be readily apparent to persons skilled in the art. Sterilization treatment regimens utilizing monochromatic germicidal, ambient temperature light, as disclosed herein, are effective in treating products/packaging of varying geometries. Thus, for example, the product and/or product package may be planar, convex, concave or an alternative geometry, e.g., a geometric combination of the foregoing geometries. The light sources may be modified to achieve desired sterilization results. Thus, for example, partially coated optical surfaces may be employed, such coated surfaces being advantageously tuned to a desired monochromatic wavelength. The use of partially coated optical surfaces may be effective in generating light that satisfies spectral intensity uniformity or intensity requirements.

Light source systems according to the present disclosure emit light over a large active area and are advantageously configured to operate at ambient temperatures. The substantially monochromatic output of these sources can be tuned to produce high spectral irradiance (watts/nm) within peaks of the process action spectra to maximize the germicidal effectiveness (or other desired process/application) as a function of the required biological objective. The range of available geometries (including coaxial sources radiating either inwardly or outwardly, and planar sources emitting from one or both sides) and the capability to independently adjust irradiance and total power provide significant flexibility in system design and allow for more efficient light delivery systems.

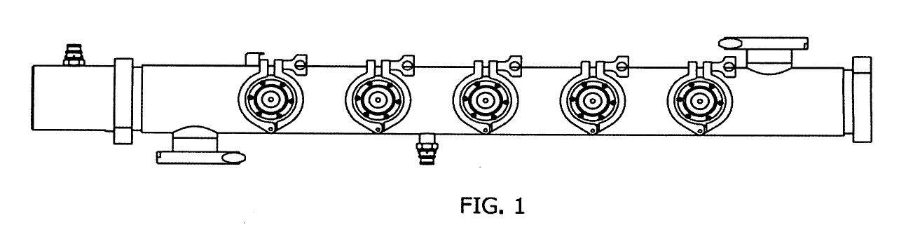

With particular reference to FIG. 1, an exemplary treatment system according to the present disclosure includes a germicidal lamp housed in a metal reactor with multiple output ports for light pipe delivery of monochromatic germicidal UV. This system may be advantageously incorporated into and operated in conjunction with an indexing feeder system. The indexing feeder system may be associated with a wide range of industrial applications, e.g., a fill and cap food and/or beverage application, a pharmaceutical application, a medicinal application, and the like. The germicidal lamp is advantageously designed to generate and emit monochromatic germicidal, ambient temperature light through a plurality of treatment ports, as depicted in FIG. 1.

According to exemplary embodiments of the present disclosure, the light source is an excimer light source that generally produces 90% of its output within a 10 nm band that can be discretely adjusted across the VUV, UV-A, UV-B and UV-C by changing the rare and/or halogen gases used. Efficiencies vary with gas mix and geometry from 10% to >30% with demonstrated input powers from <1 watt to >10 kW. The overall design and operation of exemplary light sources for use in the disclosed system are disclosed, described and depicted in commonly assigned patent application Ser. No. 09/805,610 (filed Mar. 13, 2001; published as US 2002-0177118 A1) and Ser. No. 10/661,262 (filed Sep. 12, 2003; published as US 2004-0115612 A1) (the “Prior applications”), the entire contents of which are hereby incorporated by reference in their entireties. For example, the Prior Applications disclose and describe exemplary flow patterns/arrangements for the introduction and withdrawal of cooling fluids (e.g., see tubing/hoses in FIGS. 1 and 1A thereof), exemplary treatment window designs and the like, each of which is visually apparent in FIG. 1 and/or FIG. 1A thereof.

According to exemplary embodiments of the disclosed systems, an appropriate fluid is used to maintain the light source(s) at a desired temperature and/or within a desired temperature range. Water is a preferred heat exchange medium for dissipating/absorbing heat generated through operation of the light source(s). However, alternative cooling fluids may be employed, as will be apparent to persons skilled in the art. In selecting an appropriate cooling fluid, it is desirable to select a fluid that, in use, is substantially transparent to the germicidal radiation to be passed therethrough. Of note, it is also desirable to select a fluid that is not susceptible to bubble generation and/or bubble propagation, because the presence/formation of bubbles can undesirably scatter germicidal radiation and negatively effect the sterilization efficiency and/or effectiveness of the disclosed system. Thus, precautions may be advantageously taken to minimize and/or prevent bubble formation/propagation in cooling fluid use, e.g., through the use of appropriate additives or the like.

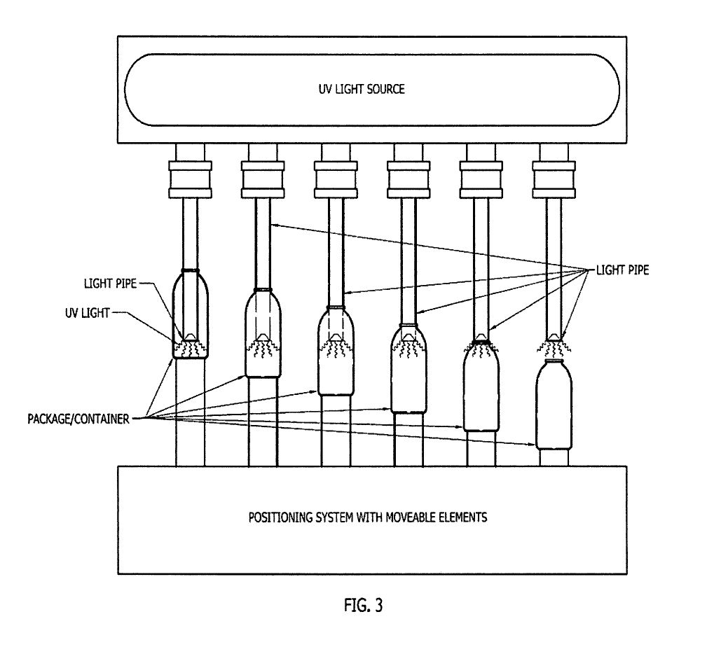

In use, packages/containers to be sterilized according to the present disclosure may be positioned in alignment with a disclosed light pipe and the light pipe may be introduced therewithin. With reference to FIG. 3, an exemplary array of packages/containers and light pipes are schematically depicted. As each light pipe is introduced into and withdrawn from an aligned package/container, the light source is energized to deliver monochromatic germicidal, ambient temperature light thereto. Germicidal light energy is thus delivered to the internal surfaces of the package/container. The light source is advantageously maintained at a substantially controlled temperature through heat transfer/heat exchange modalities, as described in the Prior Applications. As noted above, the Prior Applications are incorporated herein by reference in their entireties.

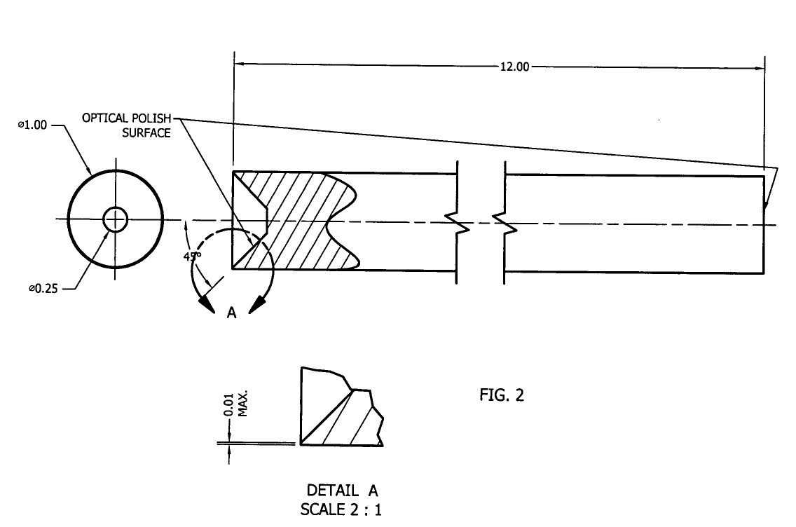

With reference to FIG. 2, an exemplary quartz light pipe according to the present disclosure is depicted. The exemplary light pipe depicted in FIG. 2 includes a polished end for substantially uniform germicidal light transmission, with or without reflective or coated surfaces, and produces a substantially uniform disc of light, whose circumference provides germicidal UV intensities at various controlled time exposures for the delivery of application-specific dosages. Further, the light sources (not visible) are positioned within a reactor housing and are advantageously maintained at a substantially constant temperature utilizing heat transfer/heat exchange modalities, as described in the Prior applications. A treatment region is defined at the disc output of the quartz light pipe.

A conveyor/transport system (not visible) is advantageously provided for transporting and indexing products through treatment region. According to exemplary embodiments of the present disclosure, the conveyor may advance the products through treatment region in a fixed orientation relative to the light source(s). Alternatively, in may be desirable to include structure(s) and/or mechanism(s) that are effective to cause repositioning of the products relative to the light source(s) as they pass through the treatment region. For example, in the case of material thickness and/or irregularly shaped containers, it may be desirable to effect rotation of the products at one or more points within the treatment region. Effective structure(s) and/or mechanism(s) for effecting reorientation of the products within the treatment region may be associated with the conveyor, with the upper and/or lower housings, or a combination thereof. The repositioning of the products may be effected in a substantially random fashion, e.g., by providing diverter walls or the like, or may be effected in a controlled fashion, e.g., through controlled robotics or the like. In any case, the inclusion of a repositioning mechanism may be desirable to provide efficient and reliable sterilization treatments to products of various sizes and geometries.

According to the present disclosure, a sterilization level>3 Log Removal may be achieved for bio-burdened packaging products that include a panel that may include (but are not limited to) Bacillus pumilus (spore former), Candida albican (yeast), Alicyclobacillus, Lipid and non-lipid virus, Clostridium sporogenes (anaerobic spore former), Staphylococcus aureus (vegetative Gram positive), Pseudomonas aeruginosa (vegetative Gram negative), Aspergillus niger (filamentous fungi), Mycobacterium terrae, Porcine Parvo Virus (PPV and B19), Lysteria, Salmonella. In achieving the foregoing Log Reduction, the overall performance properties of the sterilized packaging products are not materially affected.

In operating the disclosed sterilization treatment systems, numerous processing variables and/or product properties may influence the effectiveness of the sterilization treatment and/or the associated product survivability criteria (i.e., post-sterilization product performance and/or efficacy). For example, exemplary processing variables and product properties that are likely to influence appropriate/optimal sterilization results for food/beverage packaging and containers include:
Power delivery to light sources (power is directly related to the UV radiation dose delivered to products).
Treatment time (treatment time is directly related to the UV radiation dose delivered to products).
Hybrid product material to be treated (material and radius/diameter of the package/container may influence the desired/optimal UV radiation dose).
Irregular container handles may pose sterilization difficulties and may translate to a limitation on packaging designs that may be sterilized according to the present disclosure.
Toughness of future and/or specialized packaging materials (other than those referenced herein, e.g., polyethyleneterephthalate, polyethylenenapthalene, polyethylene, polypropylene, paperboard, aluminum foil laminated aseptic bags, and glass) may affect survivability and/or sterilization performance.

### Example 1

Testing was performed using a prototype sterilization system according to the present disclosure. The prototype system included a UV lamp as described in applicant's co-pending non-provisional patent application entitled “System and Method for Product Sterilization Using UV Light Source” (Ser. No. 11/056,698; filing date Feb. 11, 2005). The foregoing patent application was published as U.S. Publication No. 2005/0173652 and was previously incorporated herein by reference.

A cylindrical light pipe having a substantially flat proximal end was positioned against the surface of the UV lamp. The light pipe had a diameter of 25 mm and a length of 300 mm. Although the prototype light pipe featured a substantially flat proximal end, alternative geometries are contemplated, e.g., geometries that achieve enhanced contact/light communication between the UV lamp and the light pipe, e.g., a light pipe with a substantially concave proximal base is contemplated. The light pipe was fixedly positioned relative to the UV lamp surface using a clamping structure.

With reference to FIGS. 4a and 4b, the distal end of the light pipe utilized in the noted testing are depicted. A substantially conical cavity is formed in the distal end of the light pipe. The internal wall of the cavity is oriented at an angle of about 45° relative to the outer cylindrical wall of the light pipe. Of note, a substantially cylindrical bore is formed along the center line of the light pipe. The cylindrical bore facilitates fixturing of the light pipe for machining of the conical cavity.

For purposes of the present testing, the UV lamp was operated at two (2) test wavelengths (282 nm and 308 nm), although additional wavelengths are specifically contemplated, as described herein above. The power supplied to the UV lamp was varied using a conventional PDX generator, and the resulting light intensity (mj/cm²) was measured: (i) at the UV lamp surface, (ii) at the distal end of the light pipe, and (iii) at points on the circumference of the light discharge relative to the distal end of the light pipe. More particularly, light intensity was measured at distances of 0.5 inches and 1 inch from the distal end of the light pipe. For each distance (i.e., 0.5″ and 1″), the diameter (mm) of the light cone was also noted. Light intensities were measured using a Light Bug Monitor (IL 390C) and Detector (IL 1700) for monitoring UV output.

The test results for the noted experimental runs are set forth in Tables 1 and 2:

TABLE 1
Operation at 282 nm

| | AT | | | | | |
|---|---|---|---|---|---|---|
| | LAMP | AT LP | | | | |
| PDX | SUR- | OUT- | DIA. | AT DISC | DIA. | AT DISC |
| OUTPUT | FACE | PUT | AT 0.5″ | CIRCUM. | AT 1″ | CIRCUM. |
| Units | w/cm² | w/cm² | mm | w/cm² | mm | w/cm² |
| 5 kw | 0.265 | 0.241 | 38 | 0.199 | 50 | 0.135 |
| 6 kw | 0.318 | 0.283 | 40 | 0.241 | 53 | 0.156 |
| 6.5 kw | 0.345 | 0.317 | 41 | 0.255 | 54 | 0.187 |

TABLE 2
Operation at 308 nm

| | AT | | | | | |
|---|---|---|---|---|---|---|
| | LAMP | AT LP | | | | |
| PDX | SUR- | OUT- | DIA. | AT DISC | DIA. | AT DISC |
| OUTPUT | FACE | PUT | AT 0.5″ | CIRCUM. | AT 1″ | CIRCUM. |
| Units | w/cm² | w/cm² | mm | w/cm² | mm | w/cm² |
| 5 kw | 0.272 | 0.254 | 38 | 0.203 | 50 | 0.142 |
| 6 kw | 0.325 | 0.295 | 40 | 0.245 | 53 | 0.162 |
| 6.5 kw | 0.352 | 0.320 | 41 | 0.256 | 54 | 0.189 |

As is apparent from the results set forth herein, light is emitted from the distal end of the disclosed light pipe in a substantially uniform disc. The light pipe delivers germicidal monochromatic light in a disc-like manner and the light intensity or dosage is adjustable through adjustments/variations in operating parameters, e.g., electrical power input, wavelength and/or dosage time or duration. The disclosed sterilization systems are scalable, e.g., based on variations in the power input values and dosage time/duration with germicidal monochromatic light of a desired wavelength.

Although the present disclosure has been described with reference to exemplary embodiments thereof, it is to be understood that the disclosure is not limited thereto. For example, the light pipe may be fabricated from an alternative material (i.e., other than quartz), e.g., a polymeric material and/or lens system that is effective to transmit the requisite light energy. Thus, the systems and methods disclosed herein encompass modifications, enhancements and/or variations that will be readily apparent to persons skilled in the art, based on a review of the present disclosure, including specifically the prior applications incorporated herein by reference in their entireties.

1. A system for sterilization of a container, the system comprising:
a) a bounded volume of photon-producing gas for generating monochromatic light, with a bounded volume positioned within a fluid-tight housing that includes at least one light emitting port, and
b) at least one light pipe delivery system in communication with the at least one light emitting port.

2. A system according to claim 1, wherein the at least one light pipe delivery system is configured and dimensioned to emit a substantially uniform disc of germicidal light.

3. A system according to claim 1, wherein the at least one light pipe delivery system includes a treatment geometry that is adapted for irradiation of a container with a monochromatic light emitted from the at least one port.

4. A system according to claim 1, wherein the at least one light pipe delivery system emits a light emitting dosage that substantially corresponds to an inner treatment surface geometry of a container.

5. A system according to claim 1, wherein the at least one light pipe delivery system includes a light emitting surface geometry and treatment surface geometry selected from the group consisting of planar geometries, annular geometries, cylindrical geometries, elliptical geometries, non-symmetrical geometries, and combinations thereof.

6. A system according to claim 1, wherein the at least one light pipe delivery system is mounted to a fluid-tight housing, with the output of the at least one light pipe delivery system outwardly delivering a substantially uniform disc of light to a treatment surface.

7. A system according to claim 1, wherein the monochromatic light is generated at a wavelength that is substantially at a wavelength selected from the group consisting of 193, 222, 248, 282, 308 and 354 nm.

8. A system according to claim 1, wherein the light pipe delivery system is adapted to deliver a sterilization dosage to a container fabricated from a material selected from the group consisting of polyethyleneterephthalate, polyethylenenapthalene, polyethylene, polypropylene, paperboard, aluminum foil laminated, glass and combinations thereof.

9. A system according to claim 1, wherein the light emitting port defines a transparent portion and wherein the transparent portion is temperature-controlled by a cooling fluid that flows adjacent thereto.

10. A system according to claim 9, wherein the transparent portion is fabricated from quartz.

11. A system according to claim 1, wherein the at least one light pipe delivery system is configured and dimensioned for insertion into and removal from a container.

12. A system according to claim 1, wherein the at least one pipe delivery system is continuously inserted and removed from sequential containers.

13. A system according to claim 1, wherein a container is transported through a treatment region and receives a sterilization dose from the at least one light pipe delivery system that delivers light energy into the treatment region.

14. A system according to claim 13, further comprising a mechanism for repositioning the container relative to the at least one light pipe delivery system within the treatment region.

15. A method for sterilizing a container, comprising applying monochromatic light in a sterilization dosage to a container using at least one light pipe delivery system.

16. A method according to claim 15, wherein the container is fabricated from a material selected from the group consisting of polyethyleneterephthalate, polyethylenenapthalene, polyethylene, polypropylene, paperboard, aluminum foil laminated, glass and combinations thereof.

17. A method according to claim 15, wherein the monochromatic light is generated at a wavelength that is substantially at a wavelength selected from the group consisting of 193, 222, 248, 282, 308 and 354 nm.
